(19) United States
(12) Patent Application Publication
(10) Pub. No.: US 2007/0181829 A1
(43) Pub. Date: Aug. 9, 2007

(54) Electron-beam exposure system

(76) Inventors: Hitoshi Tanaka, Tokyo (JP); Akio Yamada, Tokyo (JP); Hiroshi Yasuda, Tokyo (JP); Yoshihisa Ooae, Tokyo (JP)

Correspondence Address: MURAMATSU & ASSOCIATES, Suite 310, 114 Pacifica, Irvine, CA, 92618, US

(21) Appl. No.: 11/650,234

(22) Filed: Jan. 5, 2007

(30) Foreign Application Priority Data
Jan. 6, 2006 (JP) 2006-001292

(51) Int. Cl. A61N 5/00

(52) U.S. Cl. 250/492.2

(57) ABSTRACT

An electron-beam exposure system includes: an electron gun; a first mask having a first opening for shaping a beam of electrons; a second mask having a second opening for shaping the beam of electrons; a stencil mask disposed below the first mask and the second mask, the stencil mask having a plurality of collective figured openings each for shaping the beam of electrons; a paralleling lens for causing the beam of electrons, which has been transmitted in, and come out of, the stencil mask, to turn into a beam of electrons which travels approximately in parallel to the optical axis; and a swing-back mask deflector for swinging back the beam of electrons which has passed through the stencil mask. N₂>N₁ may be satisfied where 1/N₁ denotes the reduction ratio of a pattern in the stencil mask to a pattern on the surface of the workpiece, and 1/N₂ denotes the reduction ratio of a pattern in the first mask and a pattern in the second mask to a pattern on the surface of the workpiece.

### CROSS-REFERENCE TO RELATED APPLICATIONS

This application is based on and claims priority of Japanese Patent Application No. 2006-1292 filed on Jan. 6, 2006, the entire contents of which are being incorporated herein by reference.

### BACKGROUND OF THE INVENTION

1. Field of the Invention

The present invention relates to an electron-beam exposure system, and specifically to an electron-beam exposure system which makes it possible to write a pattern on a workpiece with high precision by partial collective exposure.

2. Description of the Prior Art

In the case of electron-beam exposure systems of recent years, variable rectangular openings or a plurality of mask patterns are made available beforehand, and one of them is selected by beam deflection. Subsequently, the selected one is transferred to a workpiece, followed by exposure.

An exposure system of this type is an electron-beam exposure system which realizes partial collective exposure as disclosed, for example, in Japanese Unexamined Patent Application Official Gazette No. 2004-88071. Partial collective exposure is a technique as follows. One pattern is selected from a plurality of patterns arranged on a mask by beam deflection, and thus a beam is irradiated on the pattern area thus selected. Thereby, a cross-section of the beam is shaped into the shape represented by the selected pattern. Subsequently, the beam is caused to pass through the mask, and thereafter the resultant beam is deflectively swung back by a deflector provided in a posterior section of the electron-beam exposure system. The resultant beam is reduced in size with a certain reduction ratio determined according to the electro-optical system. After that, the pattern represented by the beam thus obtained is transferred to a workpiece.

The number of exposure shots needed for partial collective exposure is extremely smaller when frequently-used patterns are beforehand made available on a mask than when only variable rectangular openings are beforehand made available on the mask. This enhances throughput.

However, patterns which can be made available for partial collective exposure are limited in number. That is because the mask for partial collective exposure is formed in a limited space, for example, a 2000 μm×2000 μm area.

In contrast, Japanese Patent Official Gazette No. 2849184 proposes an electron-beam exposure system which makes it possible to increase the number of pattern types which can be formed by partial collective exposure. In the case of this type of electron-beam exposure system, three apertures or more are arranged on the optical axis. A beam of electrons is shaped into a rectangle by use of a first aperture and a second aperture. The resultant beam can be partially irradiated on a pattern in a third aperture (stencil mask).

The shaping of the beam of electrons by use of the plurality of apertures (openings) in a section preceding the stencil mask as described above makes it possible to virtually increase the number of pattern types.

Nevertheless, the exposure process carried out by use of the electron-beam exposure system with the foregoing configuration sometimes results in occurrence of a phenomenon in which an exposed pattern is different from a desired pattern.

Even in a case where, for example, a beam of electrons is deviated to a blanking area on the mask by applying a voltage to a blanking deflector in order for the beam of electrons not to be irradiated on the workpiece, an unexpected pattern happens to be formed on the workpiece in some time.

In the case of a regular blanking mechanism, the damping rate of a beam is approximately 1×10⁻⁶, and no specific problems occur when the stage moves continuously. When the stage does not move for approximately one second, however, part of a beam of electrons leaking from the opening of the stencil mask is accidentally irradiated on the workpiece. As a result, an unexpected pattern is formed.

Moreover, in the case where a beam of electrons is irradiated on a selected part of the opening in the stencil mask, line widths of the exposed pattern may be different from desired line widths in some cases. This is because the exposure system is a system for forming a pattern with fine line widths.

A general practice for increasing throughput of an electron-beam exposure system is adaptation of a method of increasing the amount of current of a beam of electrons. However, a beam of electrons is not free of a phenomenon which is termed as the Coulomb effect. This effect constitutes a cause of increase in disturbance of edge sharpness of a pattern to be formed, and a cause of distortion of the pattern. The Coulomb effect is defined as a phenomenon in which the track of a beam of electrons is twisted due to the influence of repulsive force caused by electric charges of electrons of the beam so that the beam of electrons is unfocused. The Coulomb effect is larger as the amount of the current is larger and concurrently the radius of a beam traveling in the optical lens barrel is smaller. Particularly in an electron-beam exposure system of a regular type, the influence of the Coulomb effect is larger. This is because a beam of electrons which has been transmitted in, and come out of, an opening in the stencil mask is concentrated in a narrower range as a result of the effect of a reducing lens.

### SUMMARY OF THE INVENTION

The present invention has been made taking the problems with the prior art into consideration. An object of the present invention is to provide an electron-beam exposure system which makes it possible to increase throughput of partial collective exposure, and to increase precision with which a pattern is formed.

The foregoing problems are intended to be solved by an electron-beam exposure system characterized by including an electron gun, a first mask, a second mask, a first deflector, a stencil mask, a round aperture, a second deflector, a paralleling lens, a swing-back mask deflector, and a projection lens. The electron gun emits a beam of electrons. The first mask has a first opening for shaping the beam of electrons. The second mask has a second opening for shaping the beam of electrons. The first deflector is disposed between the first mask and the second mask, and deflects the beam of electrons. The stencil mask is disposed below the first mask and the second mask, and has a plurality of collective figured openings for shaping the beam of electrons. The round aperture is disposed between the stencil mask and a workpiece. The second deflector is disposed between the second mask and the stencil mask, and deflects the beam of electrons. The paralleling lens is disposed between the stencil mask and the round aperture, and causes the beam of electrons, which has been transmitted in, and come out of, one of the collective figured openings, to turn into a beam of electrons which travels approximately in parallel to the optical axis. The swing-back mask deflector is disposed between the stencil mask and the round aperture, and swings back the beam of electrons. The projection lens is disposed between the round aperture and the workpiece, and focuses the beam of electrons on the surface of the workpiece to form an image thereon.

The electron-beam exposure system according to this embodiment may satisfy N₂>N₁, where 1/N₁ denotes the reduction ratio of a pattern in the stencil mask to a pattern on the surface of the workpiece, and 1/N₂ denotes the reduction ratio of a patterns in the first mask and a pattern in the second mask to a pattern on the surface of the workpiece. In addition, the electron-beam exposure system according to this embodiment may include a blanking deflector to be disposed between the stencil mask and the round aperture so that the blanking operation is carried out at high speed.

Moreover, the electron-beam exposure system according to this embodiment may include control means with the following functions. The control means causes the blanking deflector to blank the beam of electrons which has been transmitted in, and come out of, one of the collective figured openings in the stencil mask. Once blanking the beam of electron, the control means causes the size of the beam of electrons to be reduced to zero, and drives the mask deflector, thus causing a track of the beam of electrons to be shifted to a predetermined figured opening in the stencil mask. Thereafter, the control means causes the size of the beam of electrons to become larger than the size of the predetermined figured opening in the stencil mask, and causes the blanking operation to be disengaged. Thereby, the control means causes one of the figured openings in the stencil mask to be selected.

In the case of the present invention, one of the lenses is disposed between the stencil mask and the workpiece, and this lens causes the beam of electrons which has been transmitted in, and come out of, the stencil mask, to travel approximately in parallel to the optical axis. In addition, one of the deflectors is disposed between the stencil mask and the workpiece, and swings the beam of electrons, which has traveled approximately in parallel to the optical axis, back to the optical axis. This arrangement prevents a beam of electrons, which is going to form a stencil image after passing through the stencil mask, from crossing any other beam of electrons, which is going to form another stencil image after passing through the stencil mask. This arrangement also prevents the radius of the beam of electrons from becoming narrower. Accordingly, this makes it possible to reduce the influence of the Coulomb effect.

Moreover, in the case of the present invention, after the beam of electrons is blanked by the blanking deflector, the size of the beam of electrons is reduced to zero, and thus an opening in the stencil mask is selected. This makes it possible to prevent an unexpected pattern from being formed on the workpiece.

### BRIEF DESCRIPTION OF THE DRAWINGS

FIG. 1 is a diagram showing a configuration of an electron-beam exposure system according to the present invention.

FIG. 2 is a diagram showing tracks respectively of beams of electrons in the electron-beam exposure system according the present invention.

FIG. 3 is a diagram used for explaining a process of selecting an opening in a stencil mask.

FIG. 4 is a diagram used for explaining a blanking process which is carried out using a first mask and a second mask.

FIGS. 5A and 5B are diagrams schematically showing how a part of openings in the stencil mask is selected.

FIG. 6 is a diagram schematically showing how an opening in the stencil mask is selected.

### DESCRIPTION OF THE PREFERRED EMBODIMENT

Descriptions will be hereinafter provided for an embodiment of the present invention by referring to the drawings.

First of all, descriptions will be provided for a configuration of an electron-beam exposure system. Subsequently, descriptions will be provided for masks each including an opening for shaping a beam of electrons. Thereafter, descriptions will be provided for operations of the exposure system, mainly for an operation of causing the beam of electrons, which has been transmitted in, and come out of, a stencil mask, to travel in parallel to the optical axis, and for an operation of blanking the beam of electrons. Finally, descriptions will be provided for an electron-beam exposure method.

(Configuration of Electron-beam Exposure System)

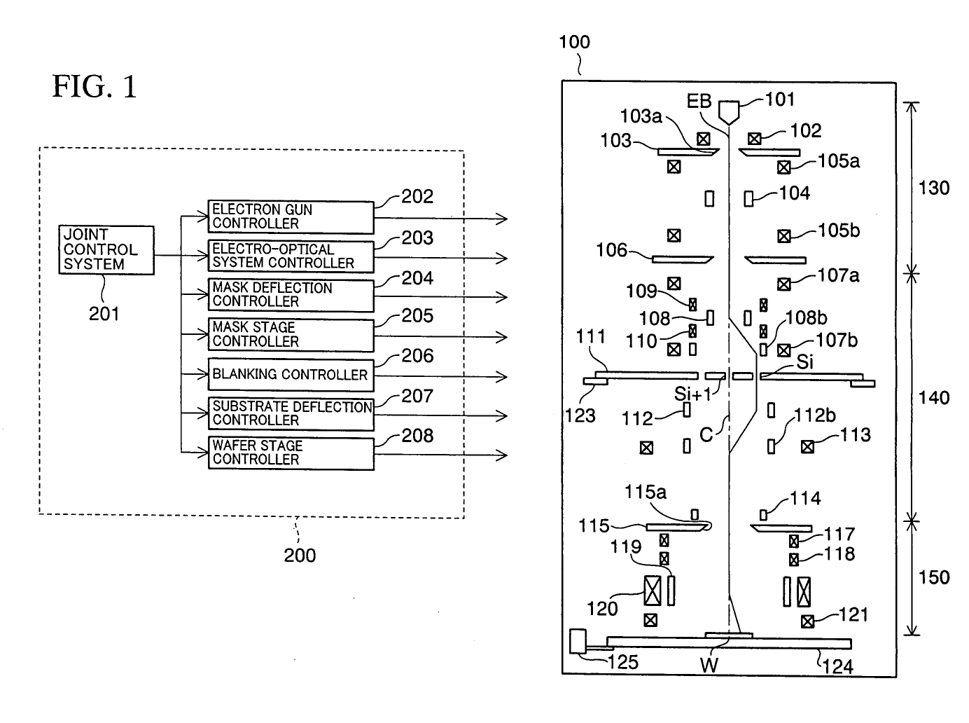

FIG. 1 shows a diagram of a configuration of the electron-beam exposure system according to this embodiment. The electron-beam exposure system is broken down into an exposer 100 and a control module 200 for controlling the exposer 100. Out of them, the exposer 100 is configured of an electron-beam generating module 130, a mask deflection module 140 and a substrate deflection module 150.

In the electron-beam generating module 130, an electron gun 101 generates a beam of electrons EB. A first electromagnetic lens 102 subjects the beam of electrons EB to a convergence effect. Thereafter, the resultant beam of electrons EB is transmitted in a rectangular aperture 103a (first opening) of a first mask 103 for shaping the beam, and thus the cross-section of the beam of electrons EB is shaped into a rectangle.

A second electromagnetic lens 105a and a third electromagnetic lens 105b focus the beam of electrons EB, which has been shaped into the rectangle, on a second mask 106 for shaping the beam, and thus the beam of electrons EB forms an image thereon. Additionally, the beam of electrons EB is deflected by a first electrostatic deflector 104 for shaping the beam of electrons into a variable rectangle. Thereafter, the beam of electrons EB thus deflected is transmitted in a rectangular aperture 106a (second opening) of the second mask 106 for shaping the beam, and comes out of the rectangular aperture 106a of the second mask 106. The beam of electrons EB is shaped by the first opening and the second opening.

After that, the beam of electrons EB is focused on a stencil mask 111 by a fourth electromagnetic lens 107a and a fifth electromagnetic lens 107b of the mask deflection module 140, and thus forms an image thereon. Additionally, the beam of electrons EB is deflected to a specific pattern Si, which has been formed in the stencil mask 111, by a second electrostatic deflector 108. Thus, the cross-sectional form of the deflected beam of electrons EB is shaped into the same form as the specific pattern Si has. The beam of electrons EB is deflected by a deflector 108b disposed in a vicinity of the fifth electromagnetic lens 107b in order that the beam of electrons EB is made incident on the stencil mask 111 while traveling in parallel to the optical axis.

Noted that, the stencil mask 111 is fixed to a mask stage 123, whereas the mask stage 123 is capable of moving in the horizontal plane. For this reason, in a case where a pattern Si existing in a part beyond the deflection range (beam deflection area) of the second electrostatic deflector 108 is intended to be used, the pattern Si is moved to the beam deflection area by moving the mask stage 123.

A sixth electromagnetic lens 113 is disposed below the stencil mask 111. By controlling the amount of current which flows to the sixth electromagnetic lens 113, this lens plays a role of causing the beam of electrons EB to travels in parallel to the optical axis near a shielding plate 115.

The beam of electrons EB which has passed through, and come out of, the stencil mask 111 is swung back to the optical axis C by a deflection effect of a third electrostatic deflector 112. A deflector 112b is disposed near the sixth electromagnetic lens 113. The deflector 112b deflects the beam of electrons EB so as for the beam of electrons EB to travel on the optical axis once the beam of electrons EB gets back onto the optical axis.

The mask deflection module 140 is provided with a first correction coil 109 and a second correction coil 110. The correction coils 109 and 110 correct aberration of the deflection of the beam, which is caused by the first to third electrostatic deflectors 104, 108 and 112.

Subsequently, the beam of electrons EB passes through a round aperture 115a of the shielding plate 115 constituting the substrate deflection module 150. The beam of electrons EB which has passed through the round aperture 115a is projected on the substrate by a projection electromagnetic lens 121. Thereby, an image representing the pattern of the stencil mask 111 is transferred to the substrate with a predetermined reduction ratio, that is, a reduction ratio of 1/10.

The substrate deflection module 150 is provided with a fourth electrostatic deflector 119 and an electromagnetic deflector 120. The beam of electrons EB is deflected by these deflectors 119 and 120, and thus the image representing the pattern of the stencil mask 111 is projected to a predetermined place in the substrate.

Furthermore, the substrate deflection module 150 is provided with a third correction coil 117 and a fourth correction coil 118 for correcting aberration of the deflection of the beam of electrons EB on the substrate.

On the other hand, the control module 200 includes an electron gun controller 202, an electro-optical system controller 203, a mask deflection controller 204, a mask stage controller 205, a blanking controller 206 and a substrate deflection controller 207. Out of these controllers, the electron gun controller 202 controls the electron gun 101. Thereby, the electron gun controller 202 controls an acceleration voltage applied to the beam of electrons EB, conditions for emitting the beam of electrons EB, and the like concerning the beam of electrons EB. In addition, the electro-optical system controller 203 controls the amount of current flowing to each of the electromagnetic lenses 102, 105a, 105b, 107a, 107b, 113 and 121 as well as the like. Thereby, the electro-optical system controller 203 adjusts magnifications, focal positions and the like of the electro-optical system in which these electromagnetic lenses are constructed. The blanking controller 206 controls a voltage applied to a blanking deflector 114. Thereby, the blanking controller 206 deflects the beam of electrons EB, which has been generated before starting the exposure, to the top of the shielding plate 115. Thus, the blanking controller 206 prevents the beam of electrons EB from being irradiated on the substrate before the exposure.

The substrate deflection controller 207 controls a voltage applied to the fourth electrostatic deflector 119 and the amount of current flowing to the electromagnetic deflector 120. Thereby, the substrate deflection controller 207 deflects the beam of electrons EB to a predetermined place in the substrate. The foregoing controllers 202 to 207 are jointly controlled by a joint control system 201 such as a workstation.

(Masks)

Rectangular openings are provided respectively to the first mask 103 and the second mask 106. The openings are 600 μm×600 μm in size, for example. By contrast, openings each representing figures of fine elements and openings each representing wiring patterns (collectively referred to as collective figured openings) are arranged in the stencil mask 111. In addition, a minute pattern requiring its precision (for example, a pattern for forming a gate of a transistor, which is 30 μm×1 μm in size) is arranged in the stencil mask 111. This pattern is transferred to the top of the workpiece, and a pattern thus formed on the workpiece is 3 μm×0.1 μm in size.

The pattern with fine line widths can be also obtained through forming a variable rectangle by use of the first mask 103 and the second mask 106.

However, the precision of the pattern with fine line widths is not so high, because the openings respectively of the first mask 103 and the second mask 106 are formed by knife edge. Moreover, the beam of electrons which is deflected when forming the variable rectangle fluctuates if the voltage fluctuates. The fluctuation of the deflection also constitutes a cause of decreasing the precision with which the pattern is formed on the workpiece.

With this fact taken into consideration, a pattern obtained through forming a variable rectangle by use of the first mask 103 and the second mask 106 is used as a pattern requiring no precision, such as a pattern for wirings or for earth lines.

On the other hand, when a pattern with line widths requiring their dimensional precision is intended to be obtained, an opening formed in the stencil mask 111 is selected. Specifically, a variable rectangle is formed by use of the first mask 103 and the second mask 106, and thus the entire pattern with the line widths, which represents the variable rectangle, is selected. This makes it possible to select an opening having high dimensional precision, which is formed in the stencil mask 111, and to thus form a pattern with high precision.

The electron-beam exposure system according to this embodiment is characterized in that N₂>N₁ is satisfied where 1/N₁ denotes the reduction ratio of a pattern in the stencil mask 111 to a pattern on the surface of the workpiece (hereinafter referred to as a “stencil mask reduction ratio”), and 1/N₂ denotes the reduction ratio of a pattern in the first mask 103 and a pattern in the second mask 106 to a pattern on the surface of the workpiece (hereinafter referred to as a “variable rectangle beam reduction ratio). For example, the variable rectangle beam reduction ratio is set at 1/50, and the stencil mask reduction ratio is set at 1/10. The setting of these reduction ratios in this manner makes it possible to increase the dimensional precision of a pattern obtained by the exposure, even if the edge roughness and the taper angles of a rectangular opening formed in the first mask 103 and the second mask 106 are not so precise as the edge roughness and the taper angles of a rectangular opening formed in the stencil mask 111.

(Operation of Exposure System)

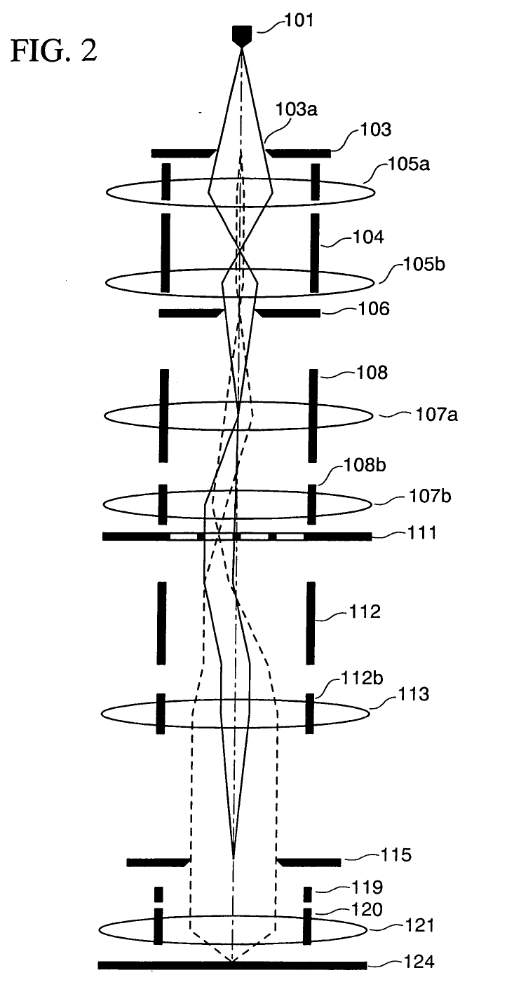

FIG. 2 is diagram showing tracks respectively of a crossover image and a mask image in the electron-beam exposure system shown in FIG. 1. In FIG. 2, a track (represented by solid lines) starting from the electron gun 101 denotes a track of the crossover image, and a track represented by dashed lines denotes a track of the mask image.

In FIG. 2, the beam of electrons emitted from the electron gun 101 is irradiated on the first mask 103. The first mask 103 is provided with the single rectangular opening 103a. An image of the opening is obtained with the beam of electrons thus irradiated. This image of the opening is formed on the second mask 106 by two lenses (the electromagnetic lenses 105a and 105b for converging the beam of electrons shaped into the rectangle.) A place of image formation on the second mask 106 is controlled by the deflector 104 (the first deflector). After passing through the opening of the second mask 106, the beam of electrons forms an image on the stencil mask 111 by two lenses (the electromagnetic lenses 107a and 107b for converging the beam of electrons shaped into the rectangle) placed in the section posterior to the second mask 106. The beam of electrons which has passed through the stencil mask 111 is swung back to the optical axis by the swing-back mask deflector 112. Subsequently, the paralleling lens 113 for paralleling the beam of electrons to the optical axis causes the beam of electrons to travel approximately in parallel to the optical axis. The resultant beam of electrons is projected to the top of the workpiece, which is placed on the stage 124, by the projection electromagnetic lens 121 (the projection lens). A place on the workpiece, where the beam of electrons forms an image, is determined by the fourth electromagnetic deflector 119 and the electrostatic deflector 120.

(Paralleling of Beam of Electrons)

The electron-beam exposure system according to this embodiment is characterized in that the electromagnetic lens 113 is disposed in the section posterior to the stencil mask 111. The electromagnetic lens 113 is that for causing the beam of electrons, which has been transmitted in, and come out of, the opening of the stencil mask 111, to travel in parallel to the optical axis near the round aperture 115a.

In the case of the prior art, once the beam of electrons is transmitted in, and comes out of, the opening in the stencil mask 111, the beam of electrons is crossed by use of two lenses. Subsequently, the beam of electrons forms an image. This practice makes the beam of electrons narrower in width, and shortens the distance between each two neighboring electrons. This makes each two neighboring electrons susceptible to each other, and the Coulomb effect makes it impossible for electrons to converge. This causes the beam of electrons to be unfocused.

In general, as current density (density of electrons) becomes larger, stronger Coulomb force works among electrons, and this force makes electrons repulse one another. This causes the beam of electrons to be unfocused.

In the case of this embodiment, the beam of electrons forms the image representing the pattern on the workpiece without crossing the beam of electrons after the beam of electrons transmits in, and comes out of, the stencil mask 111. This prevents the distance between each two neighboring electrons from be narrower, and inhibits the beam of electrons from being unfocused due to the Coulomb effect. This makes it possible to form the pattern on the workpiece with higher precision.

The foregoing descriptions have been provided chiefly for the optical image of the mask represented by the dashed lines in FIG. 2. The crossover image (represented by the solid lines in FIG. 2) starting from the electron gun 101 is formed in the following manner. Specifically, a first crossover image starting from the electron gun 101 is formed in a vicinity of the center of the first electrostatic deflector 104 (hereinafter also referred to as a “variable shaping electrostatic deflector) by use of the second electromagnetic lens 105a. Subsequently, the crossover image is sequentially formed by the lenses 105b, 107b and 113. A crossover image as a final product is formed in the round aperture 115a.

The illumination optical system of this kind is named after a person's name Koehler, and is termed as Koehler illumination. Koehler illumination is an illumination method essential for evenly illuminating the mask image on the surface of the workpiece or for evenly illuminating the stencil mask image. An image based on the image formed in the vicinity of the center of the variable shaping electrostatic deflector 104 is always formed in the same place in the round aperture 115a according to a lens's principle that the positions of the respective crossover images formed after the variable shaping electrostatic deflector 104 remain unchanged depending on the deflection electric field of the variable shaping electrostatic deflector 104. This ensures that the electron strength or the current density remains constant and unchanged in a case where the size of the variable rectangular beam is changed.

(Blanking Operation)

The electron-beam exposure system according to this embodiment is characterized in that the blanking operation is carried out to ensure that no leak beam is caused from the opening in the stencil mask 111 when the beam of electrons is blanked.

The blanking operation is carried out by the blanking deflector 114. The blanking deflector 114 is provided in order to increase the speed of blanking deflection.

When an opening in the stencil mask 111 is selected, it is likely that the beam of electrons may be transmitted in, and come out of, the opening even in a case where the beam of electrons is deviated to a blanking area on the stencil mask 111.

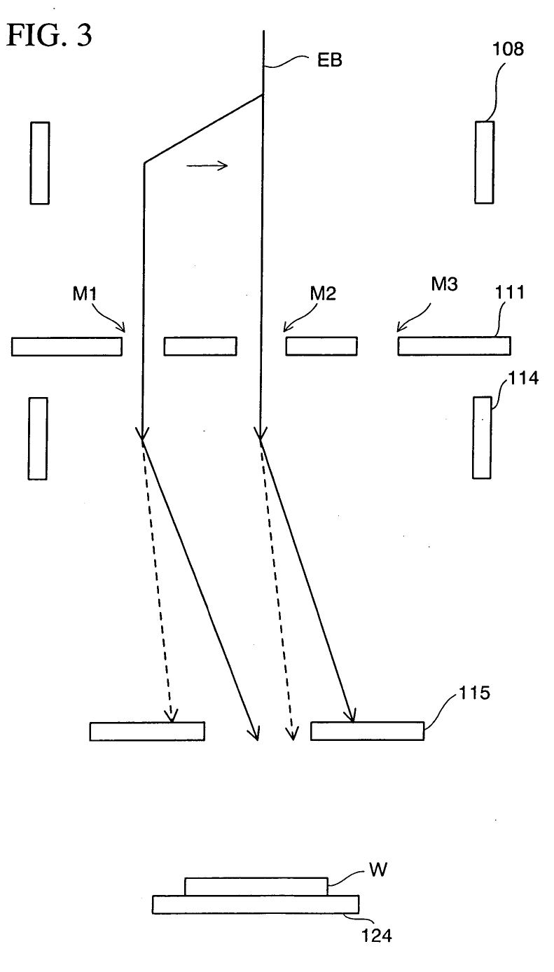

Let's discuss a case where, for example, selection of an opening M1 is followed by selection of an opening M3, as shown in FIG. 3.

No matter how the beam of electrons is deflected by the blanking deflector 114 in order not to be transmitted in, and come out of, the round aperture 115a (as shown by a left dashed line in FIG. 3), the beam of electrons has to cross an opening M2 in the middle of the shifting of the track of the beam of electrons from the opening M1 to the opening M3 with the beam of electrons irradiated with a normal amount as a result of selecting the opening M3. At that time, the beam of electrons which has been transmitted in, and come out of, the opening M2 is irradiated on a resist surface of the workpiece. This makes it likely that an unexpected pattern may be formed on the workpiece.

In the case of the electron-beam exposure system according to this embodiment, in order to take a step for coping with the foregoing problem, first of all, the beam of electrons is arranged not to be transmitted in, and come out of, the round aperture 115 by use of the blanking deflector 114 when an opening in the stencil mask 111 is intended to be selected. Subsequently, the size of the beam representing a variable rectangle is reduced in a way that the beam of electrons shaped by the rectangular opening of the first mask and the beam of electrons shaped by the rectangular opening of the second mask are not superposed on each other, the first mask and the second mask being disposed above the stencil mask. While the beam size is being reduced in such a manner, the track of the beam of electrons is shifted to a desired opening in the stencil mask 111 by driving the mask deflector 108.

Thereafter, the size of the beam representing the variable rectangle is enlarged, and the desired opening in the stencil mask 111 is obtained. Subsequently, the blanking operation is disengaged.

Because an opening in the stencil mask 111 is selected in this manner, it is not that the beam of electrons is transmitted in, or comes out of, the round aperture 115 while the track of the beam of electrons is being shifted. This makes it possible to prevent an unexpected pattern from being formed on the workpiece through exposure of the unexpected pattern to the beam of electrons, which otherwise occur.

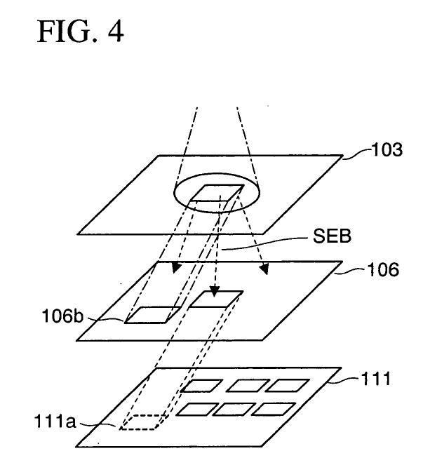

In addition, the beam of electrons can be also arranged not to be transmitted in, or come out of, an unexpected opening in the stencil mask 111 by use of the first mask and the second mask, which are disposed in the section anterior to the stencil mask 111. FIG. 4 shows a diagram used for explaining a blanking process which is carried out by use of the first mask and the second mask. When a blanking process is intended to be applied to the beam of electrons which has been transmitted, and come out of, the opening of the first mask 103, first of all, the beam of electrons is deflected by use of the deflector 104. Thereby, the beam of electrons is controlled in order to be irradiated on a blanking area 106b of the second mask 106. At this time, in addition to the beam of electrons to be deflected, beams of electrons SEB to be scattered are transmitted in, and come out of, the opening of the first mask 103. Subsequently, the scattered beams of electrons SEB (leak beams) which have been transmitted in, and come out of, the opening of the second mask 106 are deflected by use of the mask deflector 108. Thereby, the scattered beams of electrons SEB are controlled in order to be irradiated on a blanking area 111a of the stencil mask 111. The beams of electrons which have been transmitted in, and come out of, the opening of the second mask 106, are the scattered beams of electrons SEB scattered from the beam of electrons which has been transmitted in, and come out of, the opening of the first mask 103. For this reason, the energy of the scattered beams of electrons SEB which have been transmitted in, and come out of, the opening of the second mask 106 are small in amount. As a result, almost no scattered beams of electrons occur from the beam of electrons which has been transmitted in, and come of, the opening of the second mask 106. This makes it possible to prevent the leak beams from being transmitted in, and coming out of, the opening in the stencil mask 111 during the blanking operation.

The blanking process of this type to be applied to the beam of electrons is effective for preventing an unexpected pattern from being formed on the workpiece while the stage 124 is not being moved.

In the case of the electron-beam exposure system according to this embodiment, as described above, the paralleling lens 113 is disposed below the stencil mask 111 in order for the beam of electrons to travel in parallel to the optical axis after having been transmitted in, and come out of, the opening in the stencil mask 111. For this reason, the beam of electrons which has been transmitted in, and come out of, the opening in the stencil mask 111 need not be reduced in size by use of a reduction lens. This prevents the distance of each two neighboring electrons from becoming shorter.

This makes it possible to minimize the Coulomb effect, and to decrease the unfocused condition of the beam of electrons.

In addition, when an opening in the stencil mask 111 is intended to be selected, the beam of electrons representing the variable rectangle is arranged not to be transmitted in, or come out of, the round aperture 115 by use of the blanking deflector 114, and thereafter the beam of electrons is reduced in size. Subsequently, a desired opening in the stencil mask 111 is selected by driving the mask deflector 108.

Because the opening in the stencil mask 111 is selected in this manner, no beam of electrons is transmitted in, or comes out of, the round aperture 115 while the track of the beam of electrons is being shifted. This makes it possible to prevent an unexpected pattern from being formed on the workpiece through exposure of the unexpected pattern to the beam of electrons, which would otherwise occur.

Furthermore, the first mask 103 and the second mask 106 are disposed above the stencil mask 111. Thus, the beam of electrons which has been transmitted in, and come out of, the opening of the first mask 103 is arranged to be irradiated on the blanking area 106b on the second mask 106 in the blanking process using the deflector 104 for shaping the beam of electrons into a variable rectangle. In addition, scattered beams of electrons are arranged to be irradiated on the blanking area 111a on the stencil mask 111 by use of the mask deflector 108. This inhibits the leak beams from passing through an undesired opening in the stencil mask 111, and accordingly inhibits the beam of electrons from being irradiated on the workpiece during the blanking operation. This makes it possible to inhibit an unexpected exposure.

(Electron-beam Exposure Method)

Descriptions will be provided hereinafter for an exposure method using the electron-beam exposure system which has been described above.

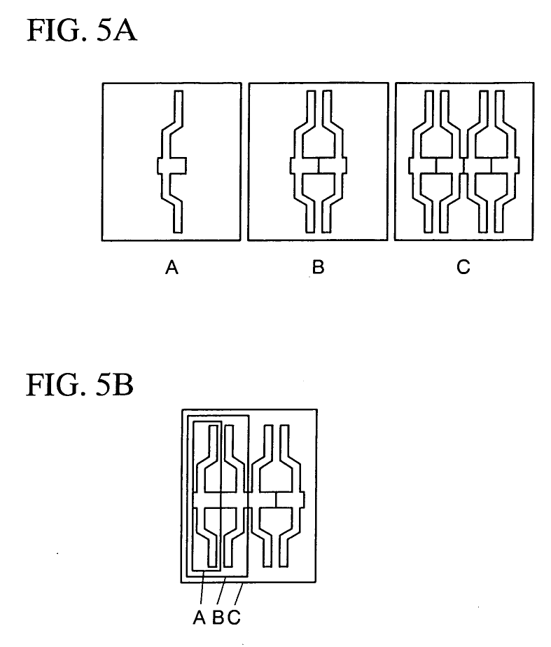

In this respect, descriptions will be provided for the exposure method, citing an example of a case where one of patterns as shown in FIG. 5A are formed through exposure of the pattern to the beam of electrons. Noted that it is assumed that openings as shown in FIG. 5B are beforehand formed in the stencil mask 111.

In a case where a pattern A in FIG. 5A is intended to be formed through exposure of the pattern A to the beam of electrons, a pattern shown by reference numeral A (hereinafter referred to as a “pattern A”) out of the patterns as shown in FIG. 5B is selected. In order to select the pattern A as shown in FIG. 5B, the opening 103a of the first mask 103 and the opening 106a of the second mask 106 are optically superposed on each other, and thus the beam of electrons is shaped into the form including nothing but the pattern A as shown in FIG. 5B. The beam of electrons thus shaped is irradiated on the pattern A as shown in FIG. 5B, which is in the stencil mask 111, by driving the second deflector 108. The beam of electrons thus irradiated is shaped into the form of the pattern A as shown in FIG. 5B. Subsequently, the beam of electrons thus shaped is transmitted in, and comes out of, the opening portion of the stencil mask 111. Thereafter, the beam of electrons is controlled by the paralleling lens 113 in order to travel in parallel to the optical axis in a vicinity of the third mask 115. The beam of electrons is converged by the projection lens 121, and thus the pattern A as shown in FIG. 5B is formed on the workpiece through exposure of the pattern A to the beam of electrons.

In a case where one of the patterns B and C which are larger than the pattern A is intended to be formed, the first opening 103a and the second opening 106a are optically superposed on each other in order that the beam of electrons can be shaped into the form including nothing but the selected pattern, and thus the exposure is carried out, in common with the case where the pattern A is selected.

If, as described above, the beam of electrons is shaped by use of one of the two masks 103 and 106 having the respective rectangular openings, which are disposed in the section anterior to the stencil mask 111, this makes it possible to select a part of the openings in the stencil mask 111. This makes it possible to obtain a plurality of patterns from one of the opening patterns in the stencil mask 111, and thus to obtain the same effect as is obtained in a case where a plurality of openings are prepared beforehand.

As described above, the partial irradiation of the beam of electrons on a desired pattern of the opening patterns makes it possible to form the desired pattern on the workpiece through the exposure of the desired pattern to the beam of electrons. However, the beam of electrons which has been transmitted in, and come out of, the stencil mask 111 is a mixture including the beam of electrons shaped by edges in the stencil mask 111 and the beam of electrons shaped by the first opening 103a and the second opening 106a. It is likely that this mixture decreases the dimensional precision of the formed pattern. For this reason, in a case where higher dimensional precision is required for the line widths, the beam of electrons shaped in the form of the rectangle by the first opening 103a and the second opening 106a needs to include all of the beam of electrons shaped by the selected pattern in the stencil mask 111.

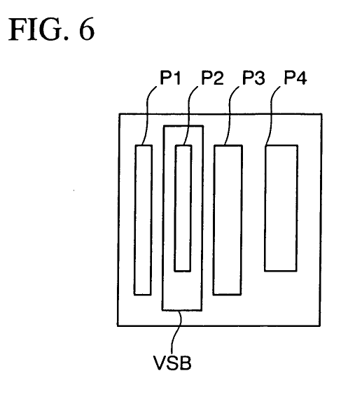

FIG. 6 shows an example where patterns each requiring dimensional precision for line widths are formed in the stencil mask 111. Patterns each requiring such precision include a 30 μm×1 μm rectangular pattern to be used, for example, to form a gate of a transistor. In a case where a pattern P2 as shown in FIG. 6 is intended to be selected, the first opening 103a and the second opening 106a are optically superposed on each other, and thus the beam of electrons is shaped into the form of a rectangle VSB in order that the shaped beam of electrons can include nothing but the pattern P2 as shown in FIG. 6. The beam of electrons shaped into the form of the pattern P2 is transmitted in, and comes out of, the opening portion of the stencil mask 111. Subsequently, the shaped beam of electrons is controlled by the paralleling lens 113 in order to travel in parallel to the optical axis in the vicinity of the third mask 115. Thereafter, the beam of electrons is converged by the projection lens 121, and thus a pattern represented by the pattern P2 is formed on the workpiece through the exposure of the pattern P2 to the beam of electrons.

The formation of the pattern through the exposure of the pattern to the beam of electrons by use of the opening which has been formed in the stencil mask 111 with high precision in this manner makes it possible to perform the exposure with higher precision.

What is claimed is:

1. An electron-beam exposure system comprising:
an electron gun for emitting a beam of electrons;
a first mask including a first opening for shaping the beam of electrons;
a second mask including a second opening for shaping the beam of electrons;
a first deflector, disposed between the first mask and the second mask, for deflecting the beam of electrons;
a stencil mask disposed below the first mask and the second mask, the stencil mask including a plurality of collective figured openings each for shaping the beam of electrons;
a round aperture disposed between the stencil mask and a workpiece;
a second deflector, disposed between the second mask and the stencil mask, for deflecting the beam of electrons;
a paralleling lens, disposed between the stencil mask and the round aperture, for causing the beam of electrons, which has been transmitted in, and come out of, one of the collective figured openings, to turn into a beam of electrons which travels approximately in parallel to the optical axis;
a swing-back mask deflector, disposed between the stencil mask and the round aperture, for swinging back the beam of electrons to the optical axis; and
a projection lens, disposed between the round aperture and the workpiece, for focusing the beam of electrons on the surface of the workpiece to form an image thereon.

2. The electron-beam exposure system according to claim 1, wherein any one of a part and all of the collective figured openings formed in the stencil mask is selected by use of the beam of electrons shaped by superposing the first opening and the second opening on each other.

3. The electron-beam exposure system according to claim 1, wherein N₂>N₁ is satisfied where 1/N₁, denotes the reduction ratio of a pattern in the stencil mask to a pattern on the surface of the workpiece, and 1/N₂ denotes the reduction ratio of a pattern in the first mask and a pattern in the second mask to a pattern on the surface of the workpiece.

4. The electron-beam exposure system according to claim 1, further comprising a blanking deflector, disposed between the stencil mask and the round aperture,
wherein a blanking operation is carried out at a high speed.

5. The electron-beam exposure system according to claim 4, further comprising a control means, wherein
the control means causes the blanking deflector to blank the beam of electrons which has been transmitted in, and come out of, one of the collective figured openings in the stencil mask,
once the beam of electrons is blanked, the control means causes the size of the beam of electrons to be reduced to zero,
the control means drives the mask deflector, and thus causes the driven mask deflector to shift a track of the beam of electrons to a predetermined figured opening in the stencil mask,
thereafter, the control means causes the size of the beam of electrons to become larger than the size of the predetermined figured opening in the stencil mask,
subsequently, the control means causes the blanking operation to be disengaged, and
thereby, the control means causes one of the figured openings in the stencil mask to be selected.
